(19) United States
(12) Patent Application Publication
(10) Pub. No.: US 2003/0095895 A1
(43) Pub. Date: May 22, 2003

(54) Device to produce a vapour

(75) Inventor: Nicholas O'Loughlin, Bondi (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sunbeam Corporation Limited, Botany (AU)

(21) Appl. No.: 10/283,485

(22) Filed: Oct. 29, 2002

(30) Foreign Application Priority Data
Nov. 19, 2001 (AU) PR8923
Nov. 19, 2001 (AU) PR8924

(51) Int. Cl. A61L 9/03

(52) U.S. Cl. 422/125; 422/306

(57) ABSTRACT

A device intended to produce a fragrant vapour. The device 10 has a plug portion 11 with electric connection pins 12 to engage a wall mounted power supply. The device 10 engages the power supply to receive electric energy therefrom as well as to be supported by the wall. The device 10 has heating elements 17 and 18 of which element 17 is mounted for angular movement so that the heat applied to a wick 15 may be varied. The device 10 has an electric circuit 30 with a timer sub-circuit 33 so that the heating elements 17 and 18 will provide heat to the wick 15 for a predetermined period of time so that a vapour is produced from the liquid contained in this wick 15.

### TECHNICAL FIELD

The present invention relates to devices to produce a vapour, and more particularly but not exclusively to domestic vaporisers to produce fragrant vapour.

### BACKGROUND OF THE INVENTION

There is known a variety of domestic devices which are electrically operated and produce a fragrant vapour. These devices are usually supported in a wall mounted power socket, with the switch of the socket being the only switch in respect of the device. That is the vaporisers are either on or off as determined by the wall switch.

A particular disadvantage of previously known devices is that the delivery rate of vapour can be higher than that required, and/or longer than is required. This is of importance as frequently the oils used are expensive.

### OBJECT OF THE INVENTION

It is the object of the present invention to overcome or substantially ameliorate the above disadvantage.

### SUMMARY OF THE INVENTION

There is disclosed herein a device to produce a vapour, said device being adapted to receive a reservoir containing a liquid from which there extends a wick, said device including:

a body to receive at least portion of the reservoir so that said wick projects into said body;

at least one heating element mounted in the body to heat the wick to aid in producing a vapour of said liquid; and

an electric circuit to deliver electric energy to said heating element to cause said heating element to heat the wick, and wherein said circuit includes a timer so that electric energy is delivered for a predetermined time, after which said element is electrically isolated so as not to heat said wick.

Preferably, said circuit includes a heating sub-circuit with an electrically operated switch which when activated electrically connects said element a supply of electric energy, and a timer sub-circuit which delivers a signal to said switch for a said period so that said switch remains activated for said period.

There is further disclosed herein a device to produce a vapour, said device being adapted to receive a reservoir containing a liquid from which there extends a wick, said device including:

a body to receive at least portion of the reservoir so that said wick projects into said body;

at least one heating element mounted in the body to heat the wick to aid in producing a vapour of said liquid, said element being mounted for movement between a first position adjacent the wick and a second position spaced from the wick so as to be displaced further from said wick relative to said first position so that the rate of vaporisation of liquid in said wick may be altered;

means to deliver electric energy to said heating element to cause said heating element to heat the wick; and

user manipulated means mounted on the body and operatively associated with the heating element so that a user may cause displacement of said element to alter the rate said liquid vaporises from said wick.

Preferably, said element is pivotally mounted in said body for angular movement between the first and second positions thereof.

Preferably, said heating element is a first heating element, and said device includes a second heating element located adjacent said wick.

Preferably, said second heating element is fixed in position relative to the wick.

Preferably, either heating element is a resistor.

### BRIEF DESCRIPTION OF THE DRAWINGS

A preferred form of the present invention will now be described by way of example with reference to the accompanying drawings wherein:

FIG. 1 is a schematic perspective view of a domestic device to produce a fragrant vapour;

FIG. 2 is a further schematic perspective view of the device of FIG. 1;

FIG. 3 is a schematic parts section top plan view of the device of FIG. 1;

FIG. 4 is a schematic parts section further top plan view of the device of FIG. 1; and

FIG. 5 is a schematic circuit diagram illustrating the electrical circuit employed in the device of FIG. 1.

### DETAILED DESCRIPTION OF THE PREFERRED EMBODIMENTS

In the accompanying drawings there is schematically depicted a device 10 to produce a fragrant vapour. The device 10 has a plug portion 11 with electric pins 12 to engage a wall mounted power supply. More particularly, the device 10 engages the power supply to receive electric energy therefrom as well as to be supported thereby on the wall.

The device 10 further includes a hollow body 13 that receives a reservoir 14. Typically, the reservoir 14 would be a bottle containing a fragrant liquid that is to be vaporised. Extending from the container 14 is a wick 15, with the container 14 projecting inwardly of the body 13 so that the wick 15 extends upwardly into the hollow body 13 so as to be received within an annular support 16.

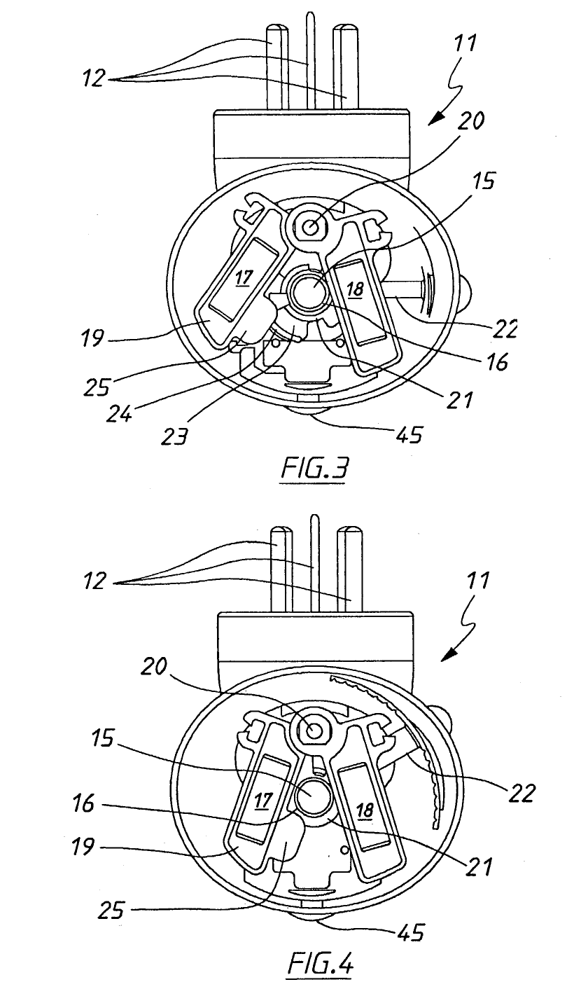

Mounted within the body 13 are heating elements 17 and 18, which are preferably electrical resistors. The element 17 is mounted for angular movement between a first position (as shown in FIG. 4) so as to be positioned adjacent the wick 15, and a second position (as shown in FIG. 3) displaced from the first position so as to be further from the wick 15. More particularly, the element 17 is mounted on a support 19, which support 19 is pivotally mounted on a post 20. Accordingly, the element 17 is angularly movable between the first and second positions thereof.

Pivotally mounted on the support 16 is an annular flange 21 from which there extends a lever 22. The lever 22 projects beyond the body 13 so that a user may grip the lever 22 to cause angular displacement of the element 17.

Extending from the annular flange 21 is a cam member 23 having an arcuate cam ridge 24 that is engaged within a track formed in the flange 25 of the support 19. Interaction between the ridge 24 and flange 25 causes pivoting of the support 19.

Preferably, the element 18 is fixed relative to the body 13 and the element 17 displaceable through 14° between its first and second positions. Preferably, the top of the wick has a temperature of about 70° C. when the element 17 is in its first position, and below 60° C. when the element 17 is in its second position.

The wick 15 projects upwardly into an upwardly extending passage 26 in the body 13 so that vapour is then distributed to the surrounding room.

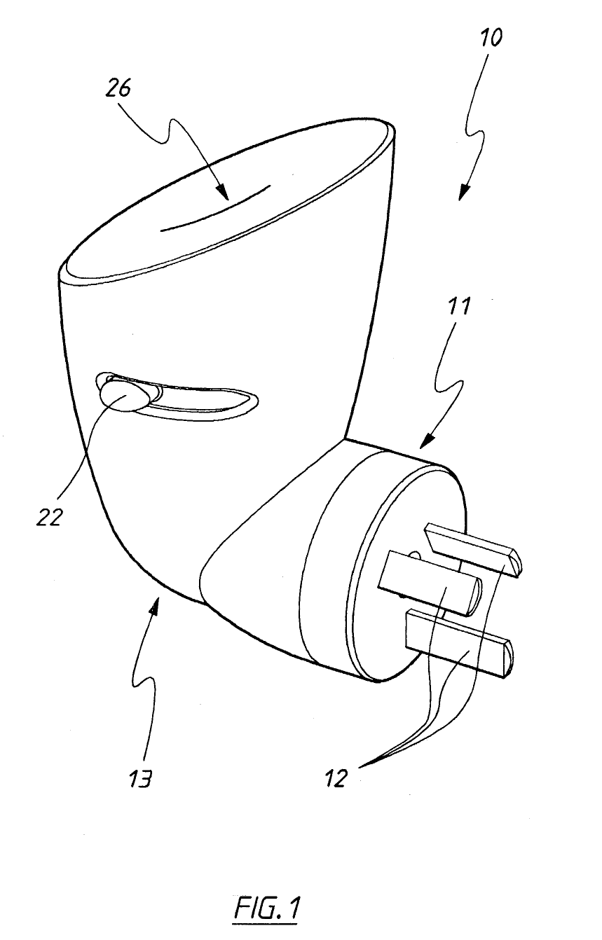

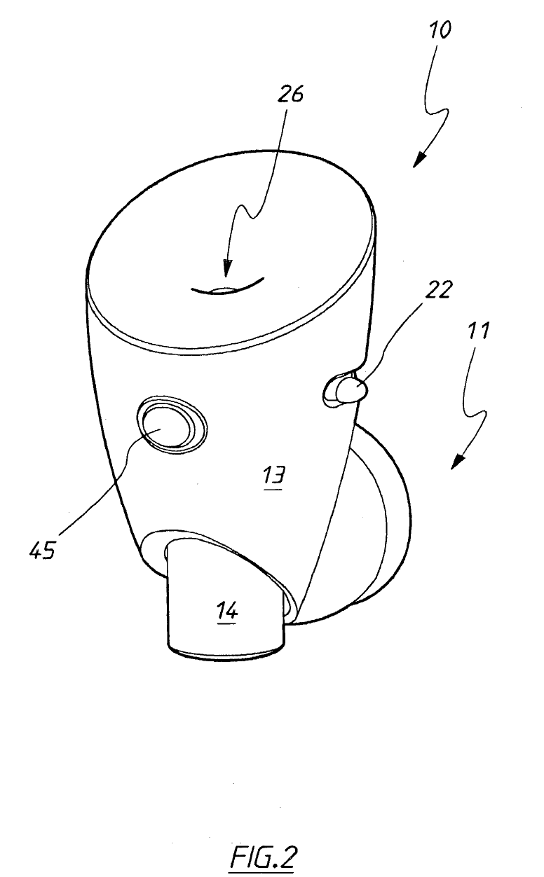

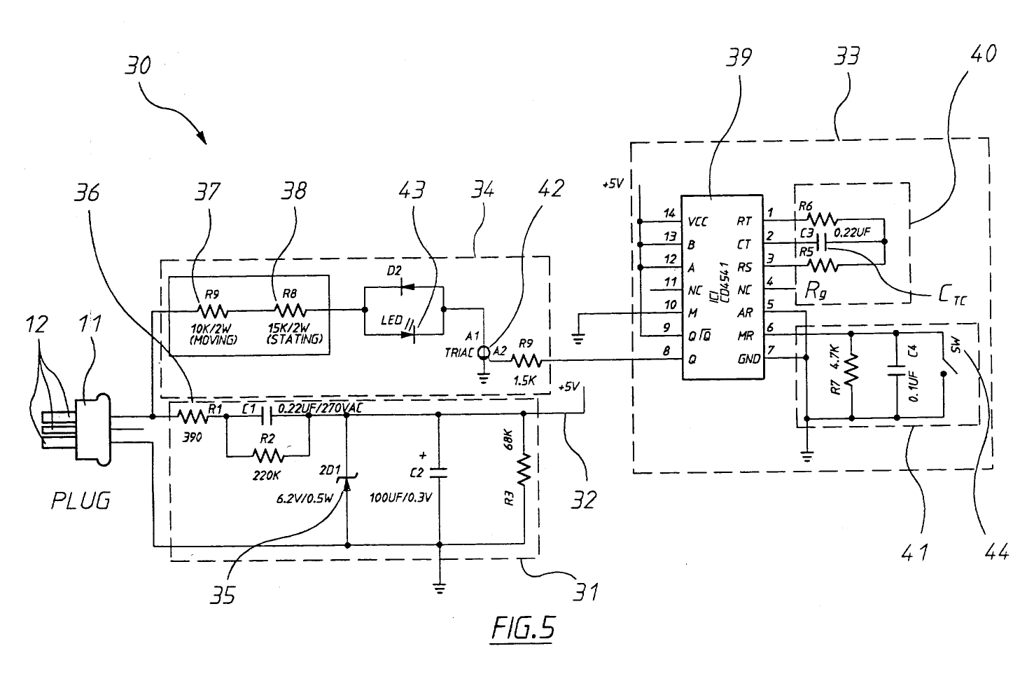

FIG. 5 is a schematic circuit diagram illustrating an electrical circuit 30 employed in the device 10 of FIG. 1. The electrical circuit 30 controls the supply of electrical power received from the wall mounted power supply through the electrical pins 12 of the plug portion 11 to the heating elements 17 and 18, which are in the form of electrical resistors 37 and 38 respectively. The electrical circuit 30 includes a heating sub-circuit 34, a timer sub-circuit 33 for providing “ON” and “OFF” signals to the heating sub-circuit 34, and a voltage reference sub-circuit 31 for providing a direct current (DC) voltage to the timer sub-circuit 33.

The resistor 37 (element 17) has a higher temperature than the resistor 38 (element 18).

The heating sub-circuit 34 includes a triode alternating current (TRIAC) switch 42 for allowing bidirectional current flow through the electrical resistors 37 and 38 while the TRIAC switch 42 receives an “ON” signal from the timer sub-circuit 33. With current flow supplied to the resistors 37 and 38, the resistors 37 and 38 heat up and supply heat to the wick 15. Alternatively the TRIAC switch 42 is “open-circuit” when an “OFF” signal is received from the timer sub-circuit 33. The heating sub-circuit 34 is also provided with a light emitting diode 43 for providing a visual indication to a user that the heating sub-circuit 34 is in operation.

The voltage reference sub-circuit 31 provides the DC voltage to the timer sub-circuit 33 upon the device 10 receiving electrical energy from the wall mounted power supply, and includes a zener diode 35 for maintaining the DC voltage at terminal 32 at 5.3V. In order to prevent damage to the device 10 and a possible fire hazard due to an over-current malfunction, the voltage reference sub-circuit 31 is preferably also provided with a resistor 36, which will fail upon an excessive current being drawn by the voltage reference sub-circuit 31.

The timer sub-circuit 33 includes an integrated circuit programmable timer 39, such as the CD4541B programmable timer available from Texas Instruments. The programmable timer 39 includes an oscillator and a counter. The oscillator is controlled by external R-C components 40, and provides pulses at a predetermined frequency ƒ to the counter. The external R-C components 40 consists of two resistors R_{TC} and R_{S}, and a capacitor C_{TC} for setting the predefined frequency ƒ as:

$$f = \frac{1}{2.3 {R}_{TC} {C}_{TC} } \qquad ( 1 )$$

Resistor R_{S} is chosen such that:

R_{S}≈2R_{TC} (2)

The timer sub-circuit 33 also includes a reset circuit 41 for resetting the counter of the programmable timer 39 when a normally open switch 44 of the reset circuit 41 is activated by a user.

Upon the timer sub-circuit 33 receiving the DC voltage from the voltage reference sub-circuit 31 or upon the counter being reset by the reset circuit 41, the counter starts counting the pulses received from the oscillator. In the embodiment illustrated, the programmable timer 39 provides an “ON” signal to the TRIAC switch 42 of the heating sub-system 34 while less than 65535 pulses have been received by the counter from the oscillator. After 65535 pulses have been received by the counter, the programmable timer 39 provides an “OFF” signal to the TRIAC switch 42, which switched off the power supply to the resistors 37 and 38, i.e. the heating elements 17 and 18.

In operation, the device 10 is plugged into the wall mounted power supply and the resistors 37 and 38 will provide heat to the wick 15 for a predetermined time period and then automatically switch off. In the preferred embodiment the predetermined time period is 4 hours. The user may reset the predetermined time period by depressing the switch 44. The led 43 and switch 44 are provided by the single lense 45 on the front of the body 13.

The claims defining the invention are as follows:

1. A device to produce a vapour, said device being adapted to receive a reservoir containing a liquid from which there extends a wick, said device including:
a body to receive at least portion of the reservoir so that said wick projects into said body;
at least one heating element mounted in the body to heat the wick to aid in producing a vapour of said liquid; and
an electric circuit to deliver electric energy to said heating element to cause said heating element to heat the wick, and wherein said circuit includes a timer so that electric energy is delivered for a predetermined time, after which said element is electrically isolated so as not to heat said wick.

2. The device of claim 1 wherein said circuit includes a heating sub-circuit with an electrically operated switch which when activated electrically connects said element a supply of electric energy, and a timer sub-circuit which delivers a signal to said switch for a said period so that said switch remains activated for said period.

3. The device of claim 1 or 2 wherein said element is mounted for movement between a first position adjacent the wick and a second position spaced from the wick so as to be displaced further from said wick relative to said first position so that the rate of vaporisation of liquid in said wick may be altered; and
user manipulated means mounted on the body and operatively associated with the heating element so that a user may cause displacement of said element to alter the rate said liquid vaporises from said wick.

4. The device of claim 3 wherein said element is pivotally mounted in said body for angular movement between the first and second positions thereof.

5. The device of claim 3 or 4 wherein said heating element is a first heating element, and said device includes a second heating element located adjacent said wick.

6. The device of claim 5 wherein said second heating element is fixed in position relative to the wick.

7. The device of claim 5 or 6 wherein either heating element is a resistor.

8. A device to produce a vapour, said device being adapted to receive a reservoir containing a liquid from which there extends a wick, said device including:
a body to receive at least portion of the reservoir so that said wick projects into said body;
at least one heating element mounted in the body to heat the wick to aid in producing a vapour of said liquid, said element being mounted for movement between a first position adjacent the wick and a second position spaced from the wick so as to be displaced further from said wick relative to said first position so that the rate of vaporisation of liquid in said wick may be altered;
means to deliver electric energy to said heating element to cause said heating element to heat the wick; and
user manipulated means mounted on the body and operatively associated with the heating element so that a user may cause displacement of said element to alter the rate said liquid vaporises from said wick.

9. The device of claim 8 wherein said element is pivotally mounted in said body for angular movement between the first and second positions thereof.

10. The device of claim 9 wherein said heating element is a first heating element, and said device includes a second heating element located adjacent said wick.

11. The device of claim 10 wherein said second heating element is fixed in position relative to the wick.

12. The device of claim 11 wherein either heating element is a resistor.
